(19) United States
(12) Patent Application Publication
(10) Pub. No.: US 2011/0130479 A1
(43) Pub. Date: Jun. 2, 2011

(54) ADHESION PROMOTER COMPOUNDS FOR OILED STEEL

(75) Inventors: Andreas Kramer, Zurich (CH); Jurgen Finter, Zurich (CH)

(73) Assignee: SIKA TECHNOLOGY AG, Baar (CH)

(21) Appl. No.: 13/054,327

(22) PCT Filed: Jul. 17, 2009
(86) PCT No.: PCT/EP2009/059213

§ 371 Date: Jan. 14, 2011

(30) Foreign Application Priority Data
Jul. 17, 2008 (EP) 08160637.8

(51) Int. Cl. C09J 163/00; C07D 303/40; C07D 303/46; C07D 407/12; C08J 9/04

(52) U.S. Cl. 521/178; 549/557; 549/553; 523/427

(57) ABSTRACT

Compounds of formula (I) in the field of heat-curing and two-component epoxy resin compositions, which may be used as adhesion promoters or adhesion promoter ingredients and methods of producing such compounds. Such compounds may be prepared as liquids. A heat-curing epoxy resin composition including at least one epoxy resin with more than one epoxy group per molecule on the average; at least one curing agent for epoxy resins, which is activated by elevated temperature; and at least one compound of formula (I).

### TECHNICAL FIELD

The invention relates to the field of heat-curing and two-component epoxy resin compositions, in particular for use as adhesives or adhesion promoters for bonding metals.

### STATE OF THE ART

Adhesives based on heat-curing and two-component epoxy resin compositions have been known for a long time in the prior art. An important application of heat-curing and two-component epoxy resin adhesives is in vehicle assembly, in particular in bonding vehicle parts in the bodyshell.

For some time, efforts have been made to improve the adhesion of these epoxy resin adhesives to challenging substrates with the help of adhesion promoters. But particularly vehicle parts made from metal having oil residues from the deep drawing process pose a special challenge for these adhesion promoters. The adhesion promoter must facilitate good adhesion on both oiled and unoiled areas of the metal. Improvement of adhesion to oiled substrates is not possible using conventional adhesion promoters.

### DESCRIPTION OF THE INVENTION

The aim of the present invention is to provide a compound which, as an adhesion promoter or an adhesion promoter ingredient, shows improvement in adhesion to oiled substrates, in particular oiled metals.

It has now surprisingly been discovered that this aim can be achieved by means of the compounds as specified in Claim 1.

A further advantage of such preferred compounds is that, thanks to their epoxy groups, when used in a heat-curing epoxy resin adhesive, they are incorporated into the adhesive during curing.

Furthermore, it has been shown that these compounds do not hinder curing of the adhesive and have good wash resistance.

It is possible to prepare such compounds as liquids which therefore can be easily dispensed and incorporated and make the use of solvents largely unnecessary.

Furthermore, such compounds exhibit extremely good wettability of the substrate, which is a necessary prerequisite for their use as adhesion promoters.

A further advantage is that the compound as specified in Claim 1 can be colored, which makes it easier to detect the compound as specified in Claim 1 after application.

Further aspects of the invention are the subject matter of other independent claims. Especially preferred embodiments of the invention are the subject matter of the dependent claims.

### Embodiments of the Invention

The present invention in a first aspect relates to a compound of formula (I):

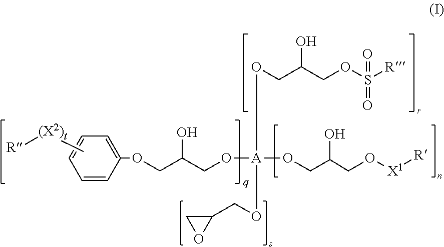

In formula (I), A stands for an (n+q+r+s)-hydric polyol A1 after removal of the (n+q+r+s) hydroxyl groups.

X¹ stands for

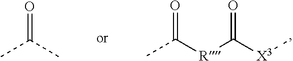

X² stands for

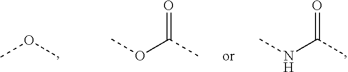

and X³ stands for O or NH.

Furthermore, R′ stands for an optionally branched, saturated or unsaturated alkyl radical with 4 to 30 C atoms, in particular 6 to 20 C atoms.

R″ stands for H or an optionally branched, saturated or unsaturated alkyl radical with 1 to 30 C atoms, in particular 6 to 20 C atoms.

R′″ stands for an alkyl radical or an alkylphenyl radical with 6 to 30 C atoms, in particular 6 to 20 C atoms.

R″″ stands for an aromatic, cycloaliphatic, or aliphatic divalent radical with 1 to 10 C atoms, in particular 2 to 8 C atoms. The aliphatic divalent radical is optionally branched, saturated or unsaturated.

Furthermore, t has a value of 0 or 1, preferably 0. The subscripts n, q, r, and s each have values from 0 to 5, provided that at least two of the subscripts n, q, and r are different from zero. In particular, s has a value of 1 or 2 and the sum (n+q+r+s) is preferably 3 or 4.

In this document, the use of the term “each independently” in connection with substituents, radicals, or groups means that substituents, radicals, or groups having the same designation can appear at the same time in the same molecule with different meanings.

The dashed lines in the formulas in this document in each case represent bonding between the respective substituents and the corresponding molecular moiety.

Here in this entire text, the prefix “poly” in “polyisocyanate,” “polyamine,” “polyol,” “polyphenol”, and “polymercaptan” indicates molecules that formally contain two or more of the respective functional groups.

“Toughener” in this document means an additive to a matrix, in particular an epoxy resin matrix, that, even for small additions of 0.1-50 wt. %, in particular 0.5-40 wt. %, causes a definite increase in toughness, and thus higher bending, tensile, shock, or impact stresses can be withstood before the matrix cracks or breaks.

“Amphiphilic block copolymer” in this document means a copolymer which contains at least one block segment miscible with epoxy resin and at least one block segment immiscible with epoxy resin. In particular, amphiphilic block copolymers are such compounds as are disclosed in WO 2006/052725 A1, WO 2006/052726 A1, WO 2006/052727 A1, WO 2006/052728 A1, WO 2006/052729 A1, WO 2006/052730 A1, and WO 2005/097893 A1, the contents of which are incorporated herein by reference.

“Adhesion promoter” in this document means a compound that improves the adhesion of an adhesive or sealant or coating to the respective substrate surface.

The polyol A1 is in particular an aliphatic, cycloaliphatic, or aromatic polyol, preferably an aliphatic polyol.

Especially suitable polyols A1 are polyols which are selected from the group consisting of pentaerythritol (=2,2-bis(hydroxymethyl)-1,3-propanediol), dipentaerythritol (=3-(3-hydroxy-2,2-bis(hydroxymethyl)propoxy-2,2-bis(hydroxymethyl)propan-1-ol), glycerol (=1,2,3-propanediol), trimethylolpropane (=2-ethyl-2-(hydroxymethyl)-1,3-propanediol), trimethylolethane (=2-(hydroxymethyl)-2-methyl-1,3-propanediol, di(trimethylolpropane) (=2,2′-oxybis(methylene)bis(2-ethylpropane-1,3-diol)), di(trimethylolethane) (=2,2′-oxybis(methylene)bis(2-methylpropane-1,3-diol)), diglycerol (=bis(2,3-dihydroxypropyl)ether), and triglycerol (=1,3-bis(2,3-dihydroxypropyl)-2-propanol).

Preferred polyols A1 are pentaerythritol, glycerol, trimethylolpropane, and trimethylolethane.

Formula (I) can be synthesized in different ways. In a first synthesis variant, the compound of formula (I) can be obtained from reaction of a polyepoxide E1 of formula (III) with a carboxylic acid of formula (IIIα) and/or a sulfonic acid of formula (IIIβ) and/or a phenol of formula (IIIγ). The subscript t′ has a value of 0 or 1.

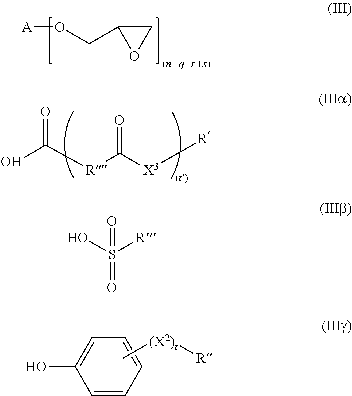

However, the compound of formula (I) must have at least one radical comprised of at least two different groups in the following list:

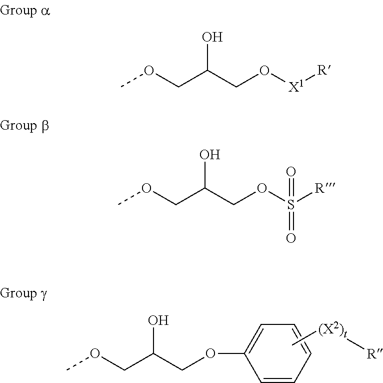

Possible combinations of the indicated groups are therefore α+β, α+γ, β+γ as well as α+β+γ.

The (n+q+r+s)-valent epoxy compounds E1 of formula (III) can be produced, for example, by reaction of polyols A1 with epichlorohydrin, as is described in the U.S. Pat. No. 5,668,227 in Example 1 for the reaction of trimethylolpropane and epichlorohydrin with tetramethylammonium chloride and sodium hydroxide solution.

The reaction of polyepoxide E1 of formula (III) with compounds of formula (IIIα) and/or compounds of formula (IIIβ) and/or compounds of formula (IIIγ) can be carried out sequentially or with a mixture of these compounds. Of course, mixtures of polyepoxides E1 of formula (III) and/or mixtures of compounds of formula (IIIα) and/or mixtures of compounds of formula (IIIβ) and/or mixtures of compounds of formula (IIIγ) can also be used.

The reaction is preferably carried out in such a way that the epoxy groups of polyepoxide E1 are present in stoichiometric excess compared with the sum of the epoxy-reactive groups of (IIIα), (IIIβ) and (IIIγ).

The reaction is preferably carried out in such a way that the compound of formula (I) has epoxy groups, typically so that 5%-95%, in particular 10%-90%, preferably 20%-80% of the sum of the epoxy groups of all the polyepoxides E1 have reacted with the sum of the epoxy-reactive groups of (IIIα), (IIIβ), and (IIIγ).

The reaction is preferably carried out in such a way that that the mole ratio of (IIIα) to (IIIβ) and/or the ratio of (IIIα) to (IIIγ) and/or the ratio of (IIIβ) to (IIIγ) is 0.05-20, in particular 0.1-20, preferably 0.2-5.

Examples of suitable carboxylic acids of formula (IIIα) are firstly:
saturated monocarboxylic acids such as pentanoic acid, hexanoic acid, heptanoic acid, octanoic acid, nonanoic acid, decanoic acid, dodecanoic acid, tetradecanoic acid, hexadecanoic acid, heptadecanoic acid, octadecanoic acid, eicosanoic acid, docosanoic acid, tetracosanoic acid, hexacosanoic acid, octacosanoic acid, triacontanoic acid or 2-ethylhexanoic acid, in particular dodecanoic acid, tetradecanoic acid, hexadecanoic acid, heptadecanoic acid, octadecanoic acid, eicosanoic acid, docosanoic acid, tetracosanoic acid, hexacosanoic acid, octacosanoic acid or triacontanoic acid.
monounsaturated or polyunsaturated monocarboxylic acids such as palmitoleic acid, oleic acid, elaidic acid, petroselinic acid, erucic acid, linoleic acid, linolenic acid, eleostearic acid, arachidonic acid, clupanodonic acid, docosahexaenoic acid, or gadoleic acid, in particular palmitoleic acid, oleic acid, linoleic acid, linolenic acid, or arachidonic acid.

Further examples of suitable carboxylic acids of formula (IIIα) are secondly dicarboxylic acid monoesters or dicarboxylic acid monoamides. Examples of such dicarboxylic acids are malonic acid, succinic acid, glutaric acid, adipic acid, trimethyladipic acid, suberic acid, azelaic acid, sebacic acid, dodecanedioic acid, maleic acid, fumaric acid, dimer fatty acid, phthalic acid, isophthalic acid, terephthalic acid, hexahydrophthalic acid, and tricarboxylic acid. When such compounds are used, the result is compounds of formula (I) in which X¹ stands for

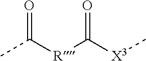

and X³ stands for O or NH.

Examples of suitable sulfonic acids of formula (IIIβ) are: aliphatic and aromatic sulfonic acids such as hexanesulfonic acid, heptanesulfonic acid, octanesulfonic acid, decanesulfonic acid, dodecanesulfonic acid, hexadecanesulfonic acid, benzenesulfonic acid, p-toluenesulfonic acid, 4-ethylbenzenesulfonic acid, 4-vinylbenzenesulfonic acid, xylenesulfonic acid, 4-octylbenzenesulfonic acid, dodecylbenzenesulfonic acid, p-xylene-2-sulfonic acid, 2-mesitylenesulfonic acid, 1-naphthalenesulfonic acid, 2-naphthalenesulfonic acid, dihexyl succinate sulfonic acid, dioctyl succinate sulfonic acid, in particular dodecanesulfonic acid, hexadecanesulfonic acid, 4-octylbenzenesulfonic acid, dodecylbenzenesulfonic acid, dihexyl succinate sulfonic acid, dioctyl succinate sulfonic acid.

Examples of suitable phenols of formula (IIIγ) are firstly alkyl-substituted monophenols such as phenol, t-butylphenol, nonylphenol, dodecylphenol, pentadecenylphenol (Cardanol).

Further examples of suitable phenols of formula (IIIγ) are secondly dihydroxybenzene monoethers resorcinol hydroquinone, and pyrocatechol, as well as esters or amides of 3- or 2-hydroxybenzoic acid or hydroxybenzamide. When such compounds are used the result is compounds of formula (I) in which

X² stands for

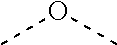

or in which X² stands for

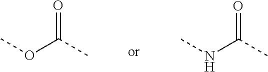

In a second variant of the synthesis, the compound of formula (I) is obtained from reaction of an (n+q+r+s)-hydric polyol A1 of formula (III′) with at least one glycidyl carboxylic acid ester of formula (III′α) and/or at least one glycidyl sulfonic acid ester of formula (III′β) and/or at least one glycidyl ether of formula (III′γ).

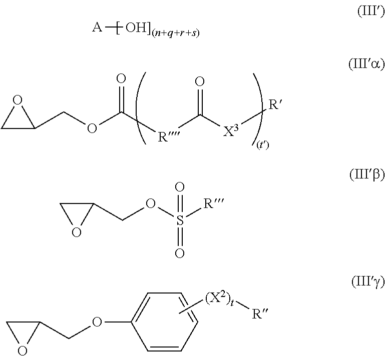

However, the compound of formula (I) must have at least one radical comprised of at least two different groups in the following list:

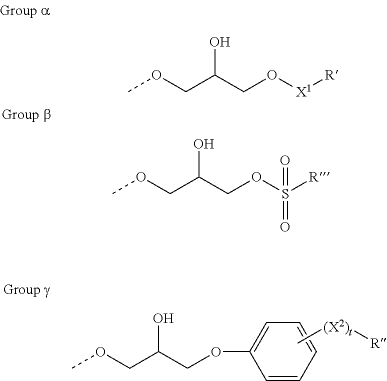

Possible combinations of the indicated groups are therefore α+β, α+γ, β+γ as well as α+β+γ.

For example, the reaction can be run for a few hours at elevated temperature such as, for example, between 100° C. and 200° C., while stirring under vacuum. Catalysts can optionally be present.

The reaction of polyol A1 of formula (III′) with a glycidyl carboxylic acid ester (III′α) and/or a glycidyl sulfonic acid ester of formula (III′β) and/or a glycidyl ether of formula (III′γ) can be carried out sequentially or with a mixture of these compounds.

Of course, mixtures of polyols A1, as well as mixtures of compounds of formula (III′α) and/or mixtures of compounds of formula (III′β) and/or mixtures of compounds of formula (III′γ) can also be used.

The reaction is preferably carried out in such a way that the sum of the epoxy groups of the compounds of formula (III′α), the compounds of formula (III′β), and the compounds of formula (III′γ) are present in stoichiometric excess compared with the hydroxyl groups of polyol A1.

Suitable compounds of formula (III′α), or compounds of formula (III′β), or compounds of formula (III′γ) can be produced, for example, by reacting the above-indicated compounds of formula (IIIα), or compounds of formula (IIIβ), or compounds of formula (IIIγ) with epichlorohydrin.

In a further aspect, the present invention relates to a heat-curing epoxy resin composition containing
at least one epoxy resin EH with more than one epoxy group per molecule on the average;
at least one curing agent B for epoxy resins, which is activated by elevated temperature; plus
at least one compound of formula (I), as described above in detail.

The epoxy resin EH with more than one epoxy group per molecule on the average is preferably a liquid epoxy resin or a solid epoxy resin. The term “solid epoxy resin” is very familiar to the person skilled in the art of epoxides, and is used in contrast to “liquid epoxy resins.” The glass transition temperature of solid resins is above room temperature, i.e., at room temperature they can be broken up into free-flowing particles.

Preferred epoxy resins have formula (II):

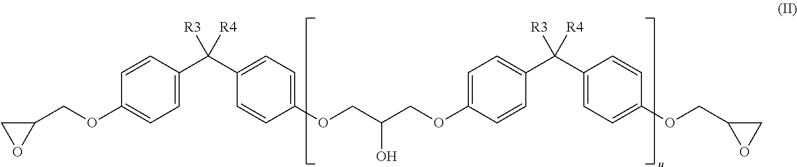

Here the substituents R³ and R⁴ each independently stand for either H or CH₃.

For solid epoxy resins, the subscript u stands for a number >1.5, in particular from 2 to 12.

Such solid epoxy resins are commercially available, for example, from Dow or Huntsman or Hexion.

Compounds of formula (II) with a subscript u from 1 to 1.5 are called semisolid epoxy resins by the person skilled in the art. For the present invention here, they are also considered as solid resins. However, epoxy resins in the narrower sense are preferred as the solid epoxy resins, i.e., where the subscript u has a value >1.5.

For liquid epoxy resins, the subscript u stands for a number less than 1. The subscript u preferably stands for a number less than 0.2.

These compounds are therefore preferably diglycidyl ethers of bisphenol A (BADGE), bisphenol F, and bisphenol A/F. Such liquid resins are available, for example, as Araldite® GY 250, Araldite® PY 304, Araldite® GY 282 (Huntsman), or D.E.R.™ 331, or D.E.R.™ 330 (Dow), or Epikote 828 (Hexion).

Furthermore, “novolacs” are suitable as epoxy resin EH. These have in particular the following formula:

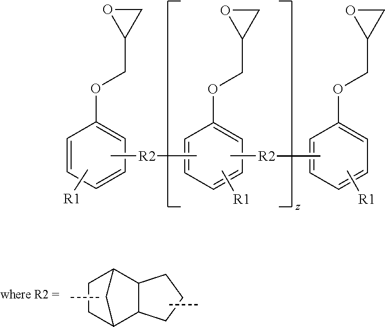

or CH₂, R1=H or methyl and z=0 to 7.

Here these can be in particular phenol or cresol novolacs (R2=CH2).

Such epoxy resins are commercially available under the trade names EPN or ECN as well as Tactix® from Huntsman or as the D.E.N.™ product line from Dow Chemical.

Epoxy resin EH preferably is a liquid epoxy resin of formula (II). In another even more preferred embodiment, the heat-curing epoxy resin composition contains at least one liquid epoxy resin of formula (II) with u<1 as well as at least one solid epoxy resin of formula (II) with u>1.5.

The proportion by weight of epoxy resin EH is 5-80 wt. %, in particular 10-75 wt. %, preferably 15-70 wt. %, based on the weight of the heat-curing epoxy resin composition.

The curing agent B for epoxy resins which is activated by elevated temperature is preferably a curing agent selected from the group consisting of dicyanodiamide, guanamines, guanidines, aminoguanidines, and derivatives thereof. Catalytically effective curing agents can also be used, such as substituted ureas such as, for example, 3-(3-chloro-4-methylphenyl)-1,1-dimethylurea (chlortoluron) or phenyl dimethylureas, in particular p-chlorophenyl-N,N-dimethylurea (monuron), 3-phenyl-1,1-dimethylurea (fenuron), or 3,4-dichlorophenyl-N,N-dimethylurea (diuron), N,N-dimethylurea, N-iso-butyl-, N′,N′-dimethylurea, adducts of diisocyanates and dialkylamines. Examples of such adducts of diisocyanates and dialkylamines are 1,1′-(hexane-1,6-diyl)bis(3,3′-dimethylurea), which is easy to obtain by reaction of hexamethylene diisocyanate (HDI) and dimethylamine, or the urea compound which is formed in addition of isophorone diisocyanate (IPDI) to dimethylamine. Compounds in the class of imidazoles, imidazolines, and amine complexes can also be used.

Curing agent B is preferably a curing agent which is selected from the group consisting of dicyanodiamide, guanamines, guanidines, aminoguanidines, and derivatives thereof; substituted ureas, in particular 3-(3-chloro-4-methylphenyl)-1,1-dimethylurea (chlortoluron) or phenyl dimethylureas, in particular p-chlorophenyl-N,N-dimethylurea (monuron), 3-phenyl-1,1-dimethylurea (fenuron), 3,4-dichlorophenyl-N,N-dimethylurea (diuron), N,N-dimethylurea, N-isobutyl-N′,N′-dimethylurea, 1,1′-(hexane-1,6-diyl)bis(3,3′-dimethylurea) as well as imidazoles, imidazole salts, imidazolines, and amine complexes.

Dicyanodiamide is particularly preferred as curing agent B.

The total proportion of curing agent B is advantageously 0.01-20 wt. %, preferably 0.1-15 wt. %, based on the weight of the heat-curing epoxy resin composition.

The amount of curing agent B for epoxy resins which is activated by elevated temperature is especially preferably 0.2-10 wt.-%, in particular 0.5-7 wt.-%, based on the weight of epoxy resin EH.

The compound of formula (I) has already been mentioned above. The proportion of the compound of formula (I) is typically 0.001-20 wt. %, in particular 0.5-15 wt. %, preferably 4-10 wt. %, based on the weight of the heat-curing epoxy resin composition.

The heat-curing epoxy resin composition advantageously contains at least one toughener D. The toughener D can be solid or liquid.

The toughener D is in particular selected from the group consisting of blocked polyurethane polymers, liquid rubbers, epoxy resin-modified liquid rubbers, block copolymers, and core/shell polymers.

In one embodiment, this toughener D is a liquid rubber D1, which is an acrylonitrile/butadiene copolymer terminated by carboxyl groups or (meth)acrylate groups or epoxy groups, or is a derivative thereof. Such liquid rubbers are commercially available, for example, under the name Hypro™ (formerly Hycar®) CTBN and CTBNX and ETBN from Nanoresins AG, Germany or Emerald Performance Materials LLC. Suitable derivatives are in particular elastomer-modified polymers having epoxy groups, such as are commercially marketed as the Polydis® product line, preferably from the Polydis® 36xx product line, by the Struktol Company (Schill & Seilacher Group, Germany) or as the Albipox product line (Nanoresins, Germany).

In a further embodiment, the toughener D is a polyacrylate liquid rubber D2 that is completely miscible with liquid epoxy resins, and only separates into microdroplets during curing of the epoxy resin matrix. Such polyacrylate liquid rubbers are available, for example, under the name 20208-XPA from Rohm and Haas.

It is clear to the person skilled in the art that mixtures of liquid rubbers can of course also be used, in particular mixtures of carboxyl-terminated or epoxy-terminated acrylonitrile/butadiene copolymers or derivatives thereof with epoxy-terminated polyurethane prepolymers.

In a further embodiment, the toughener D is a solid toughener which is an organic ion-exchanged layered mineral DE1.

The ion-exchanged layered mineral DE1 can be either a cation-exchanged layered mineral DE1c or an anion-exchanged layered mineral DE1a.

The cation-exchanged layered mineral DE1c here is obtained from a layered mineral DE1′, in which at least some of the cations have been exchanged by organic cations. Examples of such cation-exchanged layered minerals DE1c are in particular those which are mentioned in U.S. Pat. No. 5,707,439 or in U.S. Pat. No. 6,197,849.

The method for preparation of these cation-exchanged layered minerals DE1′ is also described in those patents. The layered mineral DE1′ is preferably a sheet silicate. The layered mineral DE1′ is particularly preferably a phyllosilicate as are described in U.S. Pat. No. 6,197,849, Column 2, Line 38 to Column 3, Line 5, in particular a bentonite. Layered minerals DE1′ such as kaolinite or a montmorillonite or a hectorite or an illite have been shown to be especially suitable.

At least some of the cations of the layered mineral DE1′ are replaced by organic cations. Examples of such cations are n-octylammonium, trimethyldodecylammonium, dimethyldodecylammonium, or bis(hydroxyethyl)octadecylammonium or similar derivatives of amines that can be obtained from natural fats and oils; or guanidinium cations or amidinium cations; or cations of N-substituted derivatives of pyrrolidine, piperidine, piperazine, morpholine, thiomorpholine; or cations of 1,4-diazobicyclo[2.2.2]octane (DABCO) and 1-azobicyclo[2.2.2]octane; or cations of N-substituted derivatives of pyridine, pyrrole, imidazole, oxazole, pyrimidine, quinoline, isoquinoline, pyrazine, indole, benzimidazole, benzoxazole, thiazole, phenazine, and 2,2′-bipyridine. Furthermore, cyclic amidinium cations are suitable, in particular those such as are disclosed in U.S. Pat. No. 6,197,849 in Column 3, Line 6 to Column 4, Line 67. Compared with linear ammonium compounds, cyclic ammonium compounds are distinguished by elevated thermal stability, since thermal Hofmann degradation cannot occur with them.

Preferred cation-exchanged layered minerals DE1c are familiar to the person skilled in the art under the term organoclay or nanoclay, and are commercially available, for example, under the group names Tixogel® or Nanofil® (Südchemie), Cloisite® (Southern Clay Products), or Nanomer® (Nanocor, Inc.), or Garamite® (Rockwood).

The anion-exchanged layered mineral DE1a here is obtained from a layered mineral DE1″, in which at least some of the anions have been exchanged by organic anions.

An example of such an anion-exchanged layered mineral DE1a is a hydrotalcite DE1″, in which at least some of the interlayer carbonate anions have been exchanged by organic anions.

It is also quite possible for the heat-curing epoxy resin composition to simultaneously contain a cation-exchanged layered mineral DE1c and an anion-exchanged layered mineral DE1a.

In a further embodiment, the toughener D is a solid toughener which is a block copolymer DE2. The block copolymer DE2 is obtained from an anionic or controlled free-radical polymerization of methacrylic acid ester with at least one other monomer having an olefinic double bond. Particularly preferred as a monomer having an olefinic double bond is one in which the double bond is conjugated directly with a hetero atom or with at least one other double bond. Particularly suitable monomers are selected from the group including styrene, butadiene, acrylonitrile, and vinyl acetate. Acrylate/styrene/acrylic acid (ASA) copolymers, available, for example, under the name GELOY 1020 from GE Plastics, are preferred.

Especially preferred block copolymers DE2 are block copolymers derived from methacrylic acid methyl ester, styrene, and butadiene. Such block copolymers are available, for example, as triblock copolymers under the group name SBM from Arkema.

In a further embodiment, the toughener D is a core/shell polymer DE3. Core/shell polymers consist of an elastic core polymer and a rigid shell polymer. Particularly suitable core/shell polymers consist of a core made from elastic acrylate or butadiene polymer which is enclosed in a rigid shell made from a rigid thermoplastic polymer. This core/shell structure is either formed spontaneously through separation of a block copolymer or is determined by latex polymerization or suspension polymerization followed by grafting.

Preferred core/shell polymers are “MBS polymers,” which are commercially available under the trade names Clearstrength™ from Atofina, Paraloid™ from Rohm and Haas, or F-351™ from Zeon.

Core/shell polymer particles that are optionally in suspension are especially preferred. Examples of these are GENIOPERL M23A from Wacker with a polysiloxane core and an acrylate shell, radiation crosslinked rubber particles of the NEP series manufactured by Eliokem, or Nanoprene from Lanxess or Paraloid EXL from Rohm and Haas or Kane ACE MX-120 from Kaneka.

Other comparable examples of core/shell polymers are sold under the name Albidur™ by Nanoresins AG, Germany.

Nanoscale silicates in an epoxy matrix are also suitable, such as are sold under the trade name Nanopox from Nanoresins AG, Germany.

In a further embodiment, the toughener D is a reaction product DE4 between a carboxylated solid nitrile rubber and excess epoxy resin.

In a further embodiment, the toughener D is a blocked polyurethane polymer of formula (IV).

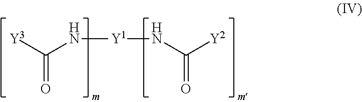

Here m and m′ each stand for numbers between 0 and 8, provided that m+m′ stands for a number from 1 to 8.

Preferably m is different from 0.

Furthermore, Y¹ stands for a linear or branched polyurethane polymer PU1 terminated by m+m′ isocyanate groups, after removal of all terminal isocyanate groups.

Y² each independently stands for a blocking group which is cleaved at a temperature above 100° C.

Y³ each independently stands for a group of formula (IV′).

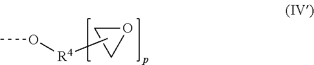

Here R⁴ stands for an aliphatic, cycloaliphatic, aromatic, or araliphatic epoxide radical containing a primary or secondary hydroxyl group, after removal of the hydroxide and epoxide groups, and p stands for the numbers 1, 2, or 3.

In this document, “araliphatic radical” means an aralkyl group, i.e., an alkyl group substituted by aryl groups (see Römpp, CD Römpp Chemie Lexikon [Römpp Chemistry Encyclopedia], Version 1, Stuttgart/New York, Georg Thieme Verlag, 1995).

Y² each independently stands in particular for a substituent selected from the group consisting of

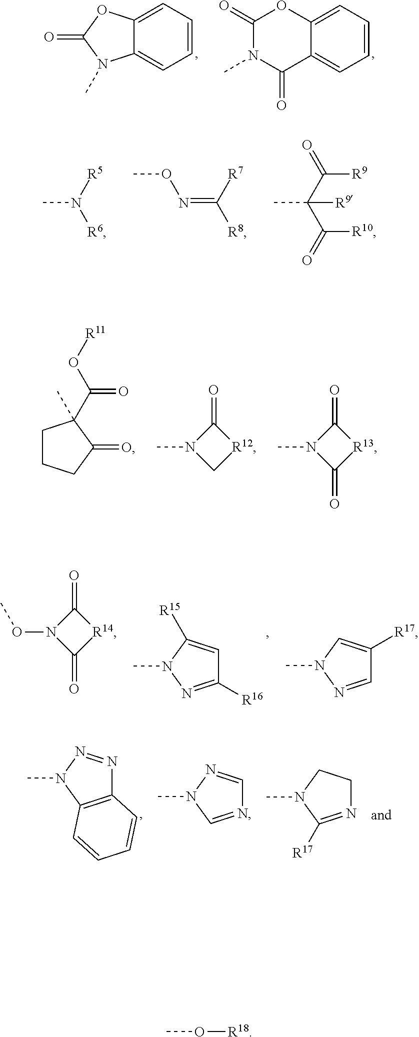

Here R⁵, R⁶, R⁷, and R⁸ each independently stand for an alkyl or cycloalkyl or aralkyl or arylalkyl group, or else R⁵ together with R⁶ or R⁷ together with R⁸ forms part of a 4- to 7-membered ring, which is optionally substituted.

Furthermore, R⁹, R^{9′}, and R¹⁰ each independently stands for an alkyl or aralkyl or arylalkyl group or for an alkyloxy or aryloxy or aralkyloxy group, and R¹¹ stands for an alkyl group.

R¹², R¹³, and R¹⁴ each independently stand for an alkylene group with 2 to 5 C atoms, which optionally has double bonds or is substituted, or for a phenylene group or for a hydrogenated phenylene group, and R¹⁵, R¹⁶, and R¹⁷ each independently stand for H or for an alkyl group or for an aryl group or an aralkyl group.

Finally, R¹⁸ stands for an aralkyl group or for a mononuclear or polynuclear substituted or unsubstituted aromatic group, which optionally has aromatic hydroxyl groups.

Phenols or bisphenols, after removal of an hydroxyl group, are in particular firstly to be considered as R¹⁸. Preferred examples of such phenols and bisphenols are in particular phenol, cresol, resorcinol, pyrocatechol, cardanol (3-Pentadecenylphenol (from cashew nutshell oil)), nonylphenol, phenols reacted with styrene or dicyclopentadiene, bisphenol-A, bisphenol-F, and 2,2′-diallylbisphenol-A.

Hydroxybenzyl alcohol and benzyl alcohol, after removal of an hydroxyl group, are in particular secondly to be considered as R¹⁸.

If R⁵, R⁶, R⁷, R⁸, R⁹, R⁹, R¹⁰, R¹¹, R¹⁵, R¹⁶ or R¹⁷ stands for an alkyl group, the latter is in particular a linear or branched C₁-C₂₀ alkyl group.

If R⁵, R⁶, R⁷, R⁸, R⁹, R^{9′}, R¹⁰, R¹⁵, R¹⁶, R¹⁷, or R¹⁸ stands for an aralkyl group, the latter group is in particular an aromatic group bonded through methylene, in particular a benzyl group.

If R⁵, R⁶, R⁷, R⁸, R⁹, R^{9′}, or R¹⁰ stands for an alkylaryl group, the latter group is in particular a C₁ to C₂₀ alkyl group bonded through phenylene such as, for example, tolyl or xylyl.

Especially preferred radicals Y² are radicals selected from the group consisting of

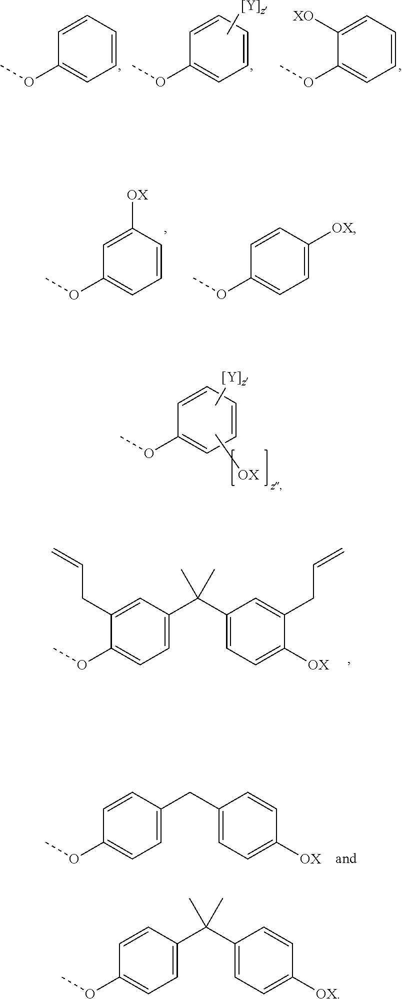

The radical Y here stands for a saturated or olefinic unsaturated hydrocarbon radical with 1 to 20 C atoms, in particular with 1 to 15 C atoms. Allyl, methyl, nonyl, dodecyl or an unsaturated C₁₅ alkyl radical with 1 to 3 double bonds are particularly preferred as Y.

The radical X stands for H or for an alkyl, aryl, aralkyl group, in particular for H or methyl.

The subscripts z′ and z″ stand for the numbers 0, 1, 2, 3, 4, or 5, provided that the sum z′+z″ stands for a number between 1 and 5.

The blocked polyurethane polymer of formula (IV) is synthesized from isocyanate group-terminated linear or branched polyurethane polymers PU1 and one or more isocyanate-reactive compounds Y²H and/or Y³H. If more than one such isocyanate-reactive compound is used, the reaction can be carried out sequentially or with a mixture of these compounds.

The reaction is carried out in such a way that the one or more isocyanate-reactive compounds Y²H and/or Y³H are used in stoichiometric amounts or in stoichiometric excess, in order to ensure that all the NCO groups are reacted.

The isocyanate-reactive compound Y³H is a monohydroxyl epoxy compound of formula (IVa).

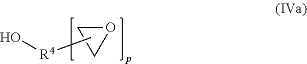

If more than one such monohydroxyl epoxy compound is used, the reaction can be carried out sequentially or with a mixture of these compounds.

The monohydroxyl epoxy compound of formula (IVa) has 1, 2, or 3 epoxy groups. The hydroxyl group of this monohydroxyl epoxy compound (IVa) can be a primary or a secondary hydroxyl group.

Such monohydroxyl epoxy compounds can, for example, be produced by reaction of polyols with epichlorohydrin. Depending on how the reaction is carried out, when polyfunctional alcohols are reacted with epichlorohydrin, the corresponding monohydroxyl epoxy compounds are also formed as byproducts in different concentrations. The latter can be isolated by means of conventional separation operations. Generally, however, it is sufficient to use the product mixture obtained in the polyol glycidylization reaction, consisting of the polyol reacted completely and partially to form the glycidyl ether. Examples of such hydroxyl-containing epoxides are butanediol monoglycidyl ethers (present in butanediol diglycidyl ethers), hexanediol monoglycidyl ethers (present in hexanediol diglycidyl ethers), cyclohexanedimethanol glycidyl ethers, trimethylolpropane diglycidyl ethers (present as a mixture in trimethylolpropane triglycidyl ethers), glycerol diglycidyl ethers (present as a mixture in glycerol triglycidyl ethers), pentaerythritol triglycidyl ethers (present as a mixture in pentaerythritol tetraglycidyl ethers). It is preferable to use trimethylolpropane diglycidyl ether, which occurs in a relatively high proportion in conventionally synthesized trimethylolpropane triglycidyl ether.

However, other similar hydroxyl-containing epoxides can also be used, in particular glycidol, 3-glycidyloxybenzyl alcohol, or hydroxymethyl cyclohexene oxide.

Also preferred is the β-hydroxy ether of formula (IVb), which is present in a proportion up to 15% in commercially available liquid epoxy resins, synthesized from bisphenol-A (R═CH₃) and epichlorohydrin, as well as the corresponding β-hydroxy ethers of formula (IVb), which are formed when bisphenol-F (R═H) or the mixture of bisphenol-A and bisphenol-F is reacted with epichlorohydrin.

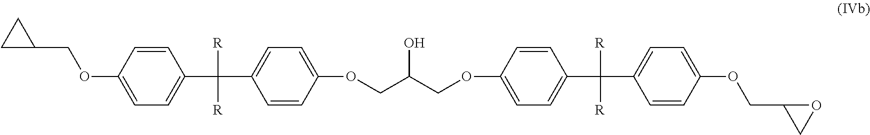

Also preferred are distillation residues produced during manufacture of high-purity distilled liquid epoxy resins. Such distillation residues have an hydroxyl-containing epoxide concentration up to three times higher than in commercially available undistilled liquid epoxy resins. Furthermore, very different epoxides with a β-hydroxy ether group, synthesized by reaction of (poly)epoxides with a substoichiometric amount of monofunctional nucleophiles such as carboxylic acids, phenols, thiols, or secondary amines, can also be used.

A trivalent radical of the following formula is particularly preferred as the radical R⁴:

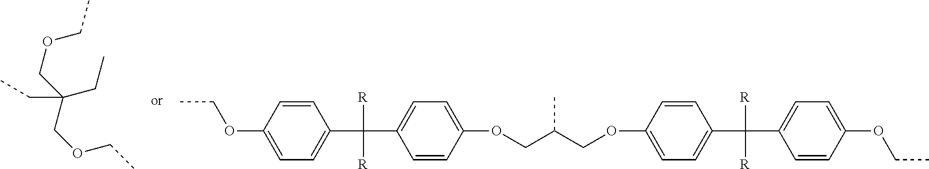

where R stands for methyl or H.

The free primary or secondary OH functional group of the monohydroxyl epoxy compound of formula (IVa) allows for efficient reaction with terminal isocyanate groups of polymers, without needing to use unusual excesses of the epoxide component.

The polyurethane polymer PU1 on which Y¹ is based can be synthesized from at least one diisocyanate or triisocyanate and at least one polymer Q_{PM} having terminal amino, thiol, or hydroxyl groups and/or one optionally substituted polyphenol Q_{PP}.

Suitable diisocyanates are, for example, aliphatic, cycloaliphatic, aromatic, or araliphatic diisocyanates, in particular commercially available products such as methylene diphenyl diisocyanate (MDI), 1,4-butane diisocyanate, hexamethylene diisocyanate (HDI), toluene diisocyanate (TDI), tolidine diisocyanate (TODI), isophorone diisocyanate (IPDI), trimethyl hexamethylene diisocyanate (TMDI), 2,5- or 2,6-bis(isocyanatomethyl)bicyclo[2.2.1]heptane, 1,5-naphthalene diisocyanate (NDI), dicyclohexylmethyl diisocyanate (H₁₂MDI), p-phenylene diisocyanate (PPDI), or m-tetramethylxylylene diisocyanate (TMXDI) as well as dimers thereof HDI, IPDI, MDI, or TDI are preferred.

Suitable triisocyanates are, for example, trimers or biurets of aliphatic, cycloaliphatic, aromatic, or araliphatic diisocyanates, in particular the isocyanurates and biurets of the diisocyanates described in the previous paragraph.

Of course, suitable mixtures of diisocyanates or triisocyanates can also be used.

Suitable polymers Q_{PM} having terminal amino, thiol, or hydroxyl groups are in particular polymers Q_{PM} having two or three terminal amino, thiol, or hydroxyl groups.

Suitable polymers Q_{PM} are in particular those such as are disclosed, for example, in WO 2008/049857 A1, in particular the Q_{PM} on page 7, line 25 to page 11, line 20, the contents of which in particular are incorporated herein by reference.

The polymers Q_{PM} advantageously have a weight per equivalent of 300-6000, in particular 600-4000, preferably 700-2200 g/equivalent of NCO-reactive groups.

Suitable polymers Q_{PM} are in particular polyoxyalkylene polyols, also called polyether polyols, hydroxy-terminated polybutadiene polyols, styrene/acrylonitrile grafted polyether polyols, polyhydroxy-terminated acrylonitrile/butadiene copolymers, polyester polyols, as well as polycarbonate polyols.

Particularly suitable as the polyphenol Q_{PP} are bisphenols, trisphenols, and tetraphenols. This means not only pure phenols but optionally also substituted phenols. The nature of the substitution can be quite diverse. In particular, this means a direct substitution on the aromatic ring to which the phenol OH group is bonded. By phenols furthermore is meant not only mononuclear aromatics but also polynuclear or condensed aromatics or heteroaromatics having phenol OH groups directly on the aromatic or heteroaromatic rings.

Bisphenols and trisphenols are especially suitable. For example, suitable bisphenols or trisphenols are 1,4-dihydroxybenzene, 1,3-dihydroxybenzene, 1,2-dihydroxybenzene, 1,3-dihydroxytoluene, 3,5-dihydroxybenzoates, 2,2-bis(4-hydroxyphenyl)propane (=bisphenol-A), bis(4-hydroxyphenyl)methane (=bisphenol-F), bis(4-hydroxyphenyl)sulfone bisphenol-S), naphthoresorcinol, dihydroxynaphthalene, dihydroxyanthraquinone, dihydroxybiphenyl, 3,3-bis(p-hydroxyphenyephthalide, 5,5-bis(4-hydroxyphenyl)hexahydro-4,7-methanoindane, phenolphthalein, fluorescein, 4,4′-[bis(hydroxyphenyl)-1,3-phenylenebis(1-methylethylidene)] (=bisphenol-M), 4,4′-bis(hydroxyphenyl)-1,4-phenylenebis(1-methylethylidene)] (=bisphenol-P), 2,2′-diallyl bisphenol-A, diphenols and dicresols synthesized by reacting phenols or cresols with diisopropylidene benzene, phloroglucinol, gallic acid esters, phenol or cresol novolacs with number of OH functional groups ranging from 2.0 to 3.5, as well as all isomers of the aforementioned compounds.

Especially suitable tougheners D optionally present in the heat-curing epoxy composition are tougheners which are amphiphilic hydroxyl group-containing block copolymers, such as are marketed under the trade name Fortegra™, in particular Fortegra™ 100, by Dow Chemical, or their reaction products with polyisocyanates and optionally other isocyanate-reactive compounds.

Especially suitable as the toughener D optionally present in the heat-curing epoxy resin composition are any of those disclosed in the following articles or patents, whose contents are incorporated here by reference: EP 0 308 664 A1, in particular formula (I), especially page 5, Line 14 to page 13, Line 24; EP 0 338 985 A1, EP 0 353 190 A1, WO 00/20483 A1, in particular formula (I), especially page 8, Line 18 to page 12, Line 2; WO 01/94492 A1, in particular the reaction products denoted as D) and E), especially page 10, Line 15 to page 14, line 22; WO 03/078163 A1, in particular the acrylate-terminated polyurethane resin denoted as B), especially page 14, Line 6 to page 14, Line 35; WO 2005/007766 A1, in particular formula (I) or (II), especially page 4, Line 5 to page 11, Line 20; EP 1 728 825 A1, in particular formula (I), especially page 3, line 21 to page 4, Line 47; WO 2006/052726 A1, in particular the amphiphilic block copolymer denoted as b), especially page 6, Line 17 to page 9, Line 10; WO 2006/052729 A1, in particular the amphiphilic block copolymer denoted as b), especially page 6, Line 25 to page 10, Line 2; T. J. Hermel-Davidock et al., J. Polym. Sci. Part B: Polym. Phys., 45, 3338-3348 (2007), in particular the ambiphilic block copolymers, especially page 3339, 2nd column to page 3341, 2nd column; WO 2004/055092 A1, in particular formula (I), especially page 7, Line 28 to page 13, Line 15; WO 2005/007720 A1, in particular formula (I), especially page 8, Line 1 to page 17, Line 10; WO 2007/020266 A1, in particular formula (I), especially page 3, Line 1 to page 11, Line 6, WO 2008/049857 A1, in particular formula (I), especially page 3, line 5 to page 6, line 20, WO 2008/049858 A1, in particular formula (I) and (II), especially page 6, line 1 to page 12, line 15, WO 2008/049859 A1, in particular formula (I), especially page 6, line 1 to page 11, line 10, WO 2008/049860 A1, in particular formula (I), especially page 3, line 1 to page 9, line 6, as well as DE-A-2 123 033, US 2008/0076886 A1, WO 2008/016889, and WO 2007/025007.

It has been shown that advantageously more than one toughener is present in the heat-curing epoxy resin composition, in particular also more than one toughener D.

The proportion of toughener D is advantageously used in an amount of 1-50 wt. %, in particular 0.5-35 wt. %, preferably 1-20 wt. %, based on the weight of the heat-curing resin epoxy composition.

In a further preferred embodiment, the heat-curing epoxy resin composition in addition contains at least one filler F. Here the filler is preferably mica, talc, kaolin, wollastonite, feldspar, syenite, chlorite, bentonite, montmorillonite, calcium carbonate (precipitated or ground), dolomite, quartz, silicic acids (pyrogenic or precipitated), cristobalite, calcium oxide, aluminum hydroxide, magnesium oxide, hollow ceramic spheres, hollow glass spheres, hollow organic spheres, glass spheres, colored pigments. As the filler F, we mean both organic coated and uncoated commercially available forms familiar to the person skilled in the art.

Another example is functionalized alumoxanes, for example as described in U.S. Pat. No. 6,322,890.

The total proportion of total filler F is advantageously 3-50 wt. %, preferably 5-35 wt. %, in particular 5-25 wt. %, based on the weight of the total heat-curing epoxy resin composition.

In a further preferred embodiment, the heat-curing epoxy resin composition contains a physical or chemical blowing agent, as is available, for example, under the trade name Expancel™ from Akzo Nobel, or Celogen™ from Chemtura, or under the trade name Luvopor® from Lehmann & Voss, The proportion of the blowing agent is advantageously 0.1-3 wt.-%, based on the weight of the heat-curing epoxy resin composition.

In another preferred embodiment, the heat-curing epoxy resin composition in addition contains at least one epoxy group-containing reactive diluent G.

These reactive diluents G are in particular:
Glycidyl ethers of monofunctional saturated or unsaturated, branched or unbranched, cyclic or open-chain C₄-C₃₀ alcohols, in particular selected from the group consisting of butyl glycidyl ether, hexyl glycidyl ether, 2-Ethylhexyl glycidyl ether, allyl glycidyl ether, tetrahydrofurfuryl and furfuryl glycidyl ether, trimethoxysilyl glycidyl ether.]
Glycidyl ethers of difunctional saturated or unsaturated, branched or unbranched, cyclic or open-chain C₂-C₃₀ alcohols, in particular selected from the group consisting of ethylene glycol glycidyl ether, butanediol glycidyl ether, hexanediol glycidyl ether, octanediol glycidyl ether, cyclohexane dimethanol diglycidyl ether, and neopentyl glycol diglycidyl ether.
Glycidyl ethers of trifunctional or polyfunctional, saturated or unsaturated, branched or unbranched, cyclic or open-chain alcohols such as epoxidized castor oil, epoxidized trimethylolpropane, epoxidized pentaerythrol, or polyglycidyl ethers of aliphatic polyols such as sorbitol, glycerol, or trimethylolpropane.
Glycidyl ethers of phenol compounds and aniline compounds, in particular selected from the group consisting of phenyl glycidyl ether, cresyl glycidyl ether, p-tert-butyl phenyl glycidyl ether, nonylphenyl glycidyl ether, 3-n-pentadecenyl glycidyl ether (from cashew nutshell oil), N,N-diglycidyl and p-aminophenyl triglycidyl [ether].
Epoxidized amines such as N,N-diglycidyl cyclohexylamine.
Epoxidized monocarboxylic acids or dicarboxylic acids, in particular selected from the group consisting of neodecanoic acid glycidyl ester, methacrylic acid glycidyl ester, benzoic acid glycidyl ester, phthalic acid diglycidyl ester, tetra- and hexahydrophthalic acid diglycidyl ester, and diglycidyl esters of dimeric fatty acids, as well as terephthalic acid glycidyl ester and trimellitic acid glycidyl ester.
Epoxidized difunctional or trifunctional, low molecular weight to high molecular weight polyether polyols, in particular polyethylene glycol diglycidyl ether or polypropylene glycol diglycidyl ether.

Hexanediol diglycidyl ether, cresyl glycidyl ether, p-tert-butylphenyl glycidyl ether, polypropylene glycol diglycidyl ether, and polyethylene glycol diglycidyl ether are especially preferred.

The proportion of the epoxy group-containing reactive diluent G is advantageously 0.1-20 wt.-%, based on the weight of the heat-curing epoxy resin composition.

In addition, the heat-curing epoxy resin composition can contain a thixotropic agent H based on a urea derivative. The urea derivative is in particular a reaction product between an aromatic monomeric diisocyanate and an aliphatic amine compound. It is also quite possible to react more than one different monomeric diisocyanates with one or more aliphatic amine compounds, or to react a monomeric diisocyanate with more than one aliphatic amine compounds. The reaction product between 4,4′-diphenylmethylene diisocyanate (MDI) and butylamine has proven to be particularly advantageous.

The urea derivative is preferably present in a carrier. The carrier can be a plasticizer, in particular a phthalate or an adipate, preferably a diisodecylphthalate (DIDP) or dioctyladipate (DOA). The carrier can also be a non-diffusing carrier. This is preferred in order to ensure the least possible migration of the unreacted ingredients after curing. Blocked polyurethane prepolymers are preferred as the non-diffusing carrier.

Preparation of Such Preferred Urea Derivatives and Carriers is Described in detail in the patent application EP 1 152 019 A1. The carrier is advantageously a blocked polyurethane prepolymer, in particular obtained by reaction of a trifunctional polyether polyol with IPDI, followed by blocking of the terminal isocyanate groups by ε-caprolactam.

The total proportion of thixotropic agent H is advantageously 0-40 wt. %, preferably 5-25 wt. %, based on the weight of the total heat-curing epoxy resin composition.

The ratio of the weight of the urea derivative to the weight of the optionally present carrier is preferably 2:98 to 50:50, in particular 5:95-25:75.

The heat-curing epoxy resin composition can include other ingredients, in particular catalysts, stabilizers, in particular heat and/or light stabilizers, thixotropic agents, plasticizers, solvents, mineral or organic fillers, blowing agents, dyes and pigments, corrosion inhibitors, surfactants, defoamers, and adhesion promoters.

Suitable plasticizers are in particular phenyl alkylsulfonic acid esters or N-butyl benzenesulfonamide, such as are commercially available as Mesamoll® or Dellatol BBS from Bayer.

Suitable stabilizers are in particular optionally substituted phenols such as BHT or Wingstay® T (Elikem), sterically hindered amines, or N-oxyl compounds such as TEMPO (Evonik)

In another aspect, the present invention relates to a two-component epoxy resin composition consisting of one resin component K1 and one curing agent component K2, where.
the resin component K1 includes at least one epoxy resin EH with more than one epoxy group per molecule on the average as well as at least one compound of formula (I); and where
The curing agent component K2 includes at least one polyamine B2 and/or polymercaptan B3.

The epoxy resin EH with more than one epoxy group per molecule on the average as well as the compound of formula (I) have been already described in detail above.

The polyamine B2 is in particular a polyamine with at least two amino groups in the form of primary or secondary amino groups.

The following polyamines are particularly suitable as polyamine B2:
aliphatic, cycloaliphatic, or arylaliphatic primary diamines, for example, ethylenediamine, 1,2-propanediamine, 1,3-propanediamine, 2-methyl-1,2-propanediamine, 2,2-dimethyl-1,3-propanediamine, 1,3-butanediamine, 1,4-butanediamine, 1,3-pentanediamine (DAMP), 1,5-pentanediamine, 1,5-diamino-2-methylpentane (MPMD), 2-butyl-2-ethyl-1,5-pentanediamine (C11-neodiamine), 1,6-hexanediamine, 2,5-dimethyl-1,6-hexanediamine, 2,2,4- and 2,4,4-trimethylhexamethylenediamine (TMD), 1,7-heptanediamine, 1,8-octanediamine, 1,9-nonanediamine, 1,10-decanediamine, 1,11-undecanediamine, 1,12-dodecanediamine, 1,2-, 1,3-, and 1,4-diaminocyclohexane, bis(4-aminocyclohexyl)methane (H₁₂-MDA), bis(4-amino-3-methylcyclohexyl)methane, bis(4-amino-3-ethykyclohexyl)methane, bis(4-amino-3,5-dimethylcyclohexyl)methane, bis(4-amino-3-ethyl-5-methylcyclohexyl)methane (M-MECA), 1-amino-3-aminomethyl-3,5,5-trimethylcyclohexane (=isophoronediamine or IPDA), 2- and 4-methyl-1,3-diaminocyclohexane and mixtures thereof, 1,3- and 1,4-bis(aminomethyl)cyclohexane, 2,5(2,6)-bis(aminomethyl)bicyclo[2.2.1]heptane (NBDA), 3(4),8(9)-bis(aminomethyl)tricyclo[5.2.1.0^{2,6}]decane, 1,4-diamino-2,2,6-trimethylcyclohexane (TMCDA), 1,8-menthanediamine, 3,9-bis(3-aminopropyl)-2,4,8,10-tetraoxaspiro[5.5]undecane, as well as 1,3- and 1,4-xylylenediamine;
ether group-containing aliphatic diamines, for example, bis(2-aminoethyl)ether, 3,6-dioxaoctane-1,8-diamine, 4,7-dioxadecane-1,10-diamine, 4,7-dioxadecane-2,9-diamine, 4,9-dioxadodecane-1,12-diamine, 5,8-dioxadecane-3,10-diamine and higher oligomers of these diamines, bis(3-aminopropyl)polytetrahydrofurans and other polytetrahydrofuran diamines with molecular weights in the range, for example, from 350 to 5200, as well as polyoxyalkylene diamines. The latter are typically amination products of polyoxyalkylene diols and can be obtained, for example, under the name Jeffamine® (from Huntsman), under the name polyetheramine (from BASF), or under the name PC Amine® (from Nitroil). Particularly suitable polyoxyalkylene diamines are Jeffamine® D-230, Jeffamine® D-400, Jeffamine® D-2000, Jeffamine® D-4000, Jeffamine®XTJ-511, Jeffamine® ED-600, Jeffamine ED-900, Jeffamine® ED-2003, Jeffamine® XTJ-568, Jeffamine® XTJ-569, Jeffamine® XTJ-523, Jeffamine® XTJ-536, Jeffamine® XTJ-542, Jeffamine® XTJ-559, Jeffamine® EDR-104, Jeffamine® EDR-148, Jeffamine® EDR-176; Polyetheramine D 230, Polyetheramine D 400 and Polyetheramine D 2000, PC Amine® DA 250, PC Amine® DA 400, PC Amine® DA 650, and PC Amine® DA 2000;
aliphatic, cycloaliphatic, or arylaliphatic triamines such as 4-aminomethyl-1,8-octanediamine, 1,3,5-tris(aminomethyl)benzene, 1,3,5-tris(aminomethyl)cyclohexane, tris(2-aminoethyl)amine, tris(2-aminopropyl)amine, tris(3-aminopropyl)amine;
polyoxyalkylene triamines, which typically are amination products of polyoxyalkylene triols and can be obtained, for example, under the trade name Jeffamine® (from Huntsman), under the name polyetheramine (from BASF), or under the name PC Amine® (from Nitroil), such as, for example, Jeffamine® T-403, Jeffamine® T-3000, Jeffamine® T-5000; Polyetheramine T403, Polyetheramine T5000; and PC Amine® TA 403, PC Amine® TA 5000;
polyamines having secondary and primary amino groups, for example, diethylenetriamine (DETA), dipropylenetriamine (DPTA), bis(hexamethylene)triamine (BHMT), 3-(2-aminoethyl)aminopropylamine, N3-(3-aminopentyl)-1,3-pentanediamine, N5-(3-aminopropyl)-2-methyl-1,5-pentanediamine, N5-(3-amino-1-ethylpropyl)-2-methyl-1,5-pentanediamine; as well as in addition the polyamines already mentioned as suitable polyamines B1;
polyamines having tertiary amino groups such as, for example, N,N′-bis(aminopropyl)piperazine, N,N-bis(3-aminopropyl)methylamine, N,N-bis(3-aminopropyl)ethylamine, N,N-bis(3-aminopropyl)propylamine, N,N-bis(3-aminopropyl)cyclohexylamine, N,N-bis(3-aminopropyl)-2-ethylhexylamine, as well as products of double cyanoethylation and subsequent reduction of fatty amines which are derived from natural fatty acids, such as N,N-bis(3-aminopropyl)dodecylamine and N,N-bis(3-aminopropyl)tallow alkylamine, which can be obtained as Triameen® Y12D and Triameen® YT (from Akzo Nobel);
polyamines having secondary amino groups such as, for example, N,N′-dibutylethylenediamine; N,N′-di(tert-butyl)ethylenediamine, N,N′-diethyl-1,6-hexanediamine, 1-[(1-methylethyl)amino]-3-[(1-methylethyl)aminomethyl)]-3,5,5-trimethylcyclohexane (Jefflink® 754 from Huntsman), N4-cyclohexyl-2-methyl-N2-(2-methylpropyl)-2,4-pentanediamine, N,N′-dialkyl-1,3-xylylenediamine, bis[4-(N-alkylamino)cyclohexyl]methane, 4,4′-trimethylene dipiperidine, N-alkylated polyetheramines, for example Jeffamine® types SD-231, SD-401, SD-404, and SD-2001 (from Huntsman);
also “polyamidoamines.” Polyamidoamine means the reaction product between a monocarboxylic acid or a polycarboxylic acid, or esters or anhydrides thereof, and an aliphatic, cycloaliphatic, or aromatic polyamine, where the polyamine is used in stoichiometric excess. Usually a “dimer fatty acid” is used as the polycarboxylic acid, and usually a polyalkyleneamine such as, for example, TETA, is used as the polyamine. Commercially available polyamidoamines are, for example, Versamid® 100, 125, 140, and 150 (from Cognis), Aradur 223, 250, and 848 (from Huntsman), Euretek® 3607, Euretek® 530 (from Huntsman), Beckopox® EH 651, EH 654, EH 655, EH 661, and EH 663 (from Cytec).

The polyamine B2 is preferably selected from the group consisting of 1,5-diamino-2-methylpentane (MPMD), 2-butyl-2-ethyl-1,5-pentanediamine (C11-neodiamine), 2,2,4- and 2,4,4-trimethylhexamethylenediamine (TMD), bis(4-amino-3-methylcyclohexyl)methane, 1-amino-3-aminomethyl-3,5,5-trimethylcyclohexane isophoronediamine or 1PDA), 1,3-bis(aminomethyl)cyclohexane, 3(4),8(9)-bis(aminomethyl)tricyclo[5.2.1.0^{2,6}]decane, 1,3-xylylenediamine, diethylenetriamine (DETA), dipropylenetriamine (DPTA), and an ether group-containing diamine derived from amination of a polyoxyalkylene diol with a molecular weight from 500 to 5000 g/mol, in particular Jeffamine® D-230 and Jeffamine® D-400.

Dimercaptans are especially preferred a polymercaptan B3. Suitable polymercaptans B3 are, for example, polymercaptoacetates of polyols. Here these are in particular polymercaptoacetates of the following polyols:
polyoxyalkylene polyols, also called polyether polyols, which are polymerization products of ethylene oxide, 1,2-propylene oxide, 1,2- or 2,3-butylene oxide, tetrahydrofuran or mixtures thereof.
hydroxy-terminated polybutadiene polyols such as, for example, those that are synthesized by polymerization of 1,3-butadiene and allyl alcohol or by oxidation of polybutadiene, as well as their hydrogenation products;
styrene/acrylonitrile-grafted polyether polyols, such as supplied, for example, by Elastogran under the name Lupranol®;
polyester polyols, synthesized for example from dihydric or trihydric alcohols reacted with organic dicarboxylic acids or their anhydrides or esters, as well as polyester polyols derived from lactones such as, for example, ε-caprolactone;
polycarbonate polyols, as can be obtained, for example, by reaction of the above-indicated alcohols (used to synthesize the polyester polyols) with dialkyl carbonates, diaryl carbonates, or phosgene;
1,2-ethanediol, diethylene glycol, 1,2-propanediol, dipropylene glycol, 1,4-butanediol, 1,5-pentanediol, 1,6-hexanediol, 1,7-heptanediol, octanediol, nonanediol, decanediol, neopentyl glycol, as well as polyols such as have been mentioned as polyols A1;
polyols such as are obtained by reduction of dimerized fatty acids.

Glycol dimercaptoacetate, trimethylolpropane trimercaptoacetate, and butanediol dimercaptoacetate are particularly preferred.

Dimercaptans of formula (V″) are the most preferred polymercaptans.

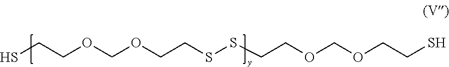

Here y stands for a number from 1 to 45, in particular from 5 to 23. The preferred molecular weights are between 800 and 7500 g/mol, in particular between 1000 and 4000 g/mol.

Such polymercaptans are commercially available as the Thiokol® LP line of Toray Fine Chemicals Co.

Mixtures of different polyamines B2 and/or polymercaptans B3 can also be used in the curing agent component K2.

The two-component epoxy resin composition additionally optionally contains at least one toughener D, at least one filler F, at least one reactive diluent G, and/or at least one thixotropic agent H, as already described previously for heat-curing epoxy resin compositions. Of course, it is clear to the person skilled in the art that there should be no ingredients within a component that could react with each other and thereby might have negative effects on the storage stability of the two-component epoxy resin composition.

Advantageous proportions of the indicated other ingredients of two-component epoxy resin compositions correspond to the proportions indicated above as advantageous for the heat-curing epoxy resin compositions.

The compound of formula (I) is exceptionally suitable as an adhesion promoter and can be widely used. This is why a further aspect of the present invention relates to use of a compound of formula (I), as indicated above, as an adhesion promoter for a substrate S1.

Preferred substrates S1 are oiled metal or an oiled metal alloy.

The compound of formula (I) is preferably used as an adhesion promoter in adhesives and coatings and particularly preferably in adhesives.

The better adhesion promotion shows that addition of a compound of formula (I) to an epoxy resin composition leads to significantly higher T-peel strengths than for the corresponding heat-curing epoxy resin composition without compounds of formula (I).

A further advantage of such compounds is that thanks to their epoxy groups, when used in a heat-curing epoxy resin adhesive, they are incorporated into the adhesive during curing. Furthermore, it has been shown that compounds of formula (I) do not hinder curing of the adhesive and have good wash resistance.

It is possible to prepare liquid compounds of formula (I) and as a result the compounds can be easily dispensed and incorporated and can make the use of solvents largely unnecessary.

Furthermore, such compounds exhibit extremely good wettability of the substrate, which is a necessary prerequisite for their use as adhesion promoters.

The compounds of formula (I) can also be used as primers or as primer ingredients. By primer is meant a primer coat which is applied to a surface and, after a certain waiting period after application (the “air-drying time”), is covered by an adhesive or sealant or coating and improves the adhesion of the thus applied adhesive or sealant or coating to the respective substrate surface.

The compound of formula (I) therefore is suitable for use as an adhesive primer for a substrate S1, in particular substrate S1 is an oiled metal or an oiled metal alloy, in particular oiled galvanized metal or metal alloy.

It is advantageous that the compound of formula (I) can be colored, which makes it easier to detect the compound as specified in Claim 1 after its application.

In another aspect, the present invention relates to use as an adhesive of a heat-curing epoxy resin composition as indicated above or a two-component epoxy resin composition as indicated above.

It has been shown that the heat-curing epoxy resin compositions described are especially suitable for use as one-component adhesives, in particular as heat-curing one-component bodyshell adhesives in vehicle assembly. Such a one-component adhesive has broad applications. Here heat-curing one-component adhesives can be realized in particular that are distinguished by high impact strength both at elevated temperatures and especially at low temperatures, in particular between 0° C. and −40° C. Such adhesives are needed for bonding heat-stable materials. “Heat-stable materials” means materials which for a cure temperature of 100° C.-220° C., preferably 120° C.-200° C., are shape-stable at least during the cure time. Here the heat-stable materials in particular are metals and plastics such as ABS, polyamide, polyphenylene ethers, composite materials such as SMC, glass fiber reinforced unsaturated polyesters, epoxy or acrylate composites. A preferred use is when at least one material is a metal. An especially preferred use is bonding of identical or different metals, in particular in bodyshells in the automobile industry. Preferred metals are especially steel, in particular electrogalvanized steel, hot-dip galvanized steel, oiled steel, Bonazinc-coated steel, and subsequently phosphatized steel as well as aluminum, in particular the types commonly used in automobile construction.

The two-component epoxy resin compositions described above can be used as an adhesive, an adhesion promoter, a sealing compound, a potting compound, a coating, a floor covering, paint, lacquer, primer or priming coat, where properties such as waterproofness, corrosion protection, chemical resistance and/or high hardness and toughness can come to the fore. It has been shown that the described two-component epoxy resin compositions are especially suitable for use as adhesives and/or as adhesion promoters. They can be used, for example, in civil engineering, in manufacture or repair of industrial goods or consumer goods, in particular vehicles.

The mix ratio between the curing agent component K1 and the resin component K2 is preferably selected so that there is a suitable ratio between the epoxy group-reactive groups of the curing agent component K1 and the epoxy groups of the resin component K2. Before curing, 0.7 to 1.5, preferably 0.9 to 1.1 equivalents of epoxy group-active NH hydrogens are suitably present per epoxy group equivalent.

The mix ratio by weight between the curing agent component K1 and the resin component K2 preferably is in the range from 1:10 to 10:1.

Both components preferably have a pasty consistency at room temperature and have comparable viscosity.

Before or during application, the two components are mixed together by means of a suitable method. Mixing can be done continuously or batchwise.

The aforementioned heat-curing epoxy resin composition, or the aforementioned two-component epoxy resin composition, can be applied to the substrate in different ways. Besides application as an adhesive bead or by spraying, they also can be applied as a film, optionally on a carrier such as netting. The aforementioned epoxy resin compositions can also contain chemical and/or physical blowing agents, leading to expansion during curing.

Preferred physical or chemical blowing agents are available, for example, under the trade name Expancel™ from Akzo Nobel, or Celogen™ from Chemtura or under the trade name Luvopor® from Lehmann & Voss. The proportion of the blowing agent is advantageously 0.1-3 wt.-%, based on the weight of the indicated epoxy resin composition.

In another aspect, the present invention relates to use of a heat-curing epoxy resin composition as indicated above or a two-component epoxy resin composition as indicated above which in addition respectively contains a chemical and/or physical blowing agent as an expandable adhesive.

In another aspect, the present invention relates to a cured composition of a heat-curing or two-component epoxy resin composition.

A cured composition of a heat-curing epoxy resin composition is obtained by heating and then crosslinking a heat-curing epoxy resin composition. Heating a heat-curing epoxy resin composition, as already described in detail above, typically is done in an oven at a temperature of 100° C.-220° C., in particular 120° C.-200° C., preferably between 130° C. and 190° C.

A cured composition of a two-component epoxy resin composition is obtained by mixing the resin component K1 and the curing agent component K2 and then crosslinking. Mixing a two-component epoxy resin composition, as described above, typically is done at a temperature of 5° C.-80° C., in particular at 10° C.-70° C.

### Examples

TABLE 1
Raw materials used.

| | |
|---|---|
| Trimethylolpropane triglycidyl ether | EMS-CHEMIE AG, Switzerland |
| (Grilonit ®V 51-31) (=“TGE”) | |
| Dimerized C18 fatty acid (Pripol 1013) | Uniquema GmbH, Germany |
| Cardanol (Cardolite ® NC-700) (=“NC”) | Cardolite Corporation, USA |
| Stearic acid (=“SA”) | Sigma-Aldrich Chemie GmbH, Switzerland |
| Bis(4-hydroxyphenyl)sulfone | Sigma-Aldrich Chemie GmbH, Switzerland |
| Bisphenol A diglycidyl ether (Araldite ® GY | Huntsman Advanced Materials GmbH, Switzerland |
| 250) (=“BADGE”) | |
| Polypropylene glycol diglycidyl ether (ED- | ADEKA Europe GmbH, Germany |
| 506) (=“ED-506”) | |
| Dicyanodiamide (=“Dicy”) | Evonik Degussa GmbH, Germany |
| Polyether polyol (Desmophen 3060BS) | Bayer MaterialScience AG, Germany |
| Isophorone diisocyanate (=1PD1) | Degussa-Hüls AG, Germany |
| Caprolactam | EMS Chemie, Switzerland |
| N-Butylamine | BASF SE, Germany |
| 4,4′-Diphenylmethylene diisocyanate (=MDI) | Bayer MaterialScience AG, Germany |
| Core/shell polymer (Zeon F-351) (=“F-351”) | Zeon Europe GmbH, Germany |
| Triphenylphosphine | Sigma-Aldrich Chemie GmbH, Switzerland |
| Dibutyltin dilaurate | Sigma-Aldrich Chemie GmbH, Switzerland |

### Preparation of Adhesion Promoter

100.0 g (0.73 eq epoxy groups) of trimethylolpropane triglycidyl ether, 65.81 g (219 mmol) Cardolite® NC-700, 20.77 g (73 mmol) stearic acid, and 0.86 g triphenylphosphine as catalyst were mixed together. The calculated epoxy group content at the start of the reaction was 3.90 eq/kg.

After 6 h of stirring at 110° C. under vacuum, the epoxide content dropped down to approximately 2.5 eq/kg and remained constant, at which point the reaction was stopped. A red viscous solution was obtained.

Different compounds 1-7 corresponding to formula (I) were prepared. BADGE was used instead of trimethylolpropane triglycidyl ether for preparation of 7. Ref1 corresponds to an unreacted mixture of trimethylolpropane triglycidyl ether, Cardolite® NC-700, and stearic acid, and thus is not according to the invention. Ref2 or Ref3 are reaction products between trimethylolpropane triglycidyl ether and respectively only steric acid or only Cardolite® NC-700, and thus are not according to the invention.

TABLE 2
Preparation of compounds of formula (I).

| | 1 | 2 | 3 | 4 | 5 |
|---|---|---|---|---|---|
| BADGE | | | | | |
| TGE | 0.730 | 0.730 | 0.730 | 0.730 | 0.730 |
| SA | 0.044 | 0.073 | 0.102 | 0.146 | 0.061 |
| NC | 0.248 | 0.219 | 0.190 | 0.146 | 0.158 |
| SA/NC | 15/85 | 25/75 | 35/65 | 50/50 | 28/72 |
| EP conversion | 40 | 40 | 40 | 40 | 30 |
| [%] | | | | | |
| | 6 | 7 | Ref1 | Ref2 | Ref3 |
| BADGE | | 0.540 | | | |
| TGE | 0.730 | | 0.730 | 0.730 | 0.730 |
| SA | 0.082 | 0.054 | 0.76 | 0.292 | — |
| NC | 0.210 | 0.162 | 0.194 | — | 0.292 |
| SA/NC | 28/72 | 25/75 | 28/72 | 100/0 | 0/100 |
| EP conversion | 40 | 40 | 0 | 40 | 40 |
| [%] | | | | | |

The proportions of the ingredients are given in Table 2 in epoxy group equivalents for the polyglycidyl ether (trimethylolpropane triglycidyl ether, or BADGE) or in phenol group equivalents for Cardolite® NC700, or carboxyl group equivalents for stearic acid.

The ratio of the proportions in carboxyl group equivalents of stearic acid to phenol group equivalents of Cardolite® NC-700 (“SA/NC”) is also shown in Table 2.

The calculated conversion of the epoxy groups of the polyglycidyl ether used (trimethylolpropane triglycidyl ether, or BADGE) for preparation of 1-7 as well as Ref1 to Ref3 (“EP Conversion”) is given in Table 2 in %. It is calculated from the sum of the phenol group and carboxyl group equivalents, divided by the epoxy group equivalents of the polyglycidyl ether used and multiplied by 100.

### Preparation of a Strength Modifier/Epoxy Resin Mixture (“Resin Mod.”)

Under vacuum and with stirring at 110° C., 123.9 g of a dimeric fatty acid, 1.1 g triphenylphosphine, and 71.3 g bis(4-hydroxyphenyl)sulfone were reacted for 5 hours with 658 g of liquid BADGE with epoxide content of 5.45 eq/kg, until a constant epoxide concentration of 2.82 eq/kg was achieved. After the end of the reaction, an additional 118.2 g of liquid BADGE was added to the reaction mixture.

### Preparation of Blocked Polyurethane Prepolymer

Under vacuum and with stirring at 90° C. in the presence of 0.08 g dibutyltin dilaurate, 600.0 g of a polyether polyol (Desmophen 3060BS; 3000 dalton; OH-number, 57 mg/g KOH) was reacted with 140.0 g IPDI to form the isocyanate-terminated prepolymer, until the isocyanate content remained constant. Then the free isocyanate groups were blocked with caprolactam (2% excess).

### Preparation of Urea Derivative Paste (“URD Paste”)

Under nitrogen and with gentle heating, 68.7 g of MD1 flakes were melted in 181.3 g of the blocked polyurethane prepolymer described above. Then 40.1 g N-butylamine dissolved in 377.1 g of the blocked prepolymer described above was added dropwise over a two-hour period, under nitrogen and with rapid stirring. After addition of the amine solution was complete, the white paste was stirred for another 30 minutes. Then after cooling down, a soft white paste was obtained which had a free isocyanate content of <0.1%.

### Preparation of Adhesive Composition

Different adhesive compositions Z1-Z9 were prepared as well as comparison compositions ZRef1-ZRef3, consisting of the ingredients as given in parts by weight in Table 3.

For each adhesive composition, the T-Peel strength (“T-Peel”) was also determined in N/mm according to DIN 53282 for a pull rate of 10 mm/min with sheet metal (0.8 mm thick), made from oiled hot-dip galvanized steel, and entered into Table 3.

TABLE 3
Weight data and the T-Peel strength for the adhesive compositions.

| | Z1 | Z2 | Z3 | Z4 | Z5 | Z6 | Z7 |
|---|---|---|---|---|---|---|---|
| Resin Mod. | 53.6 | 53.6 | 53.6 | 53.6 | 53.6 | 53.6 | 53.6 |
| ED-506 | 2.8 | 2.8 | 2.8 | 2.8 | 2.8 | 2.8 | 2.8 |
| 1 | 7.2 | | | | | | |
| 2 | | 7.2 | | | | | |
| 3 | | | 7.2 | | | | |
| 4 | | | | 7.2 | | | |
| 5 | | | | | 7.2 | | |
| 6 | | | | | | 7.2 | |
| 7 | | | | | | | 7.2 |
| URD paste | 17.4 | 17.4 | 17.4 | 17.4 | 17.4 | 17.4 | 17.4 |
| Dicy | 3.0 | 3.0 | 3.0 | 3.0 | 3.0 | 3.0 | 3.0 |
| F-351 | 6.7 | 6.7 | 6.7 | 6.7 | 6.7 | 6.7 | 6.7 |
| Filler mix | 16.5 | 16.5 | 16.5 | 16.5 | 16.5 | 16.5 | 16.5 |
| T-Peel [N/mm] | 8.8 | 10.8 | 10.6 | 9.6 | 9.0 | 10.6 | 8.9 |

| | Z8 | Z9 | ZRef1 | ZRef2 | ZRef3 |
|---|---|---|---|---|---|
| Resin Mod. | 53.6 | 53.6 | 53.6 | 53.6 | 53.6 |
| ED-506 | 2.8 | 2.8 | 2.8 | 2.8 | 2.8 |
| 2 | 5.2 | 2.7 | | | |
| Ref1 | | | 7.2 | | |
| Ref2 | | | | 3.6 | |
| Ref3 | | | | 3.6 | 7.2 |
| URD paste | 17.4 | 17.4 | 17.4 | 17.4 | 17.4 |
| Dicy | 3.0 | 3.0 | 3.0 | 3.0 | 3.0 |
| F-351 | 6.7 | 6.7 | 6.7 | 6.7 | 6.7 |
| Filler mix | 16.5 | 16.5 | 16.5 | 16.5 | 16.5 |
| T-Peel [N/mm] | 11.0 | 7.6 | 7.2 | 8.1 | 6.0 |

The results from Table 3 show that the adhesive compositions Z1-Z9, in which the polyglycidyl ether used (trimethylolpropane trigycidyl ether, or BADGE) was reacted with Cardolite® NC-700 and stearic acid, have a better T-Peel strength than ZRef1, for which the polyglycidyl ether used, Cardolite® NC-700, and stearic acid were added to an unreacted mixture.

It is also obvious from Table 3 that the adhesive compositions Z1-Z7, in which the polyglycidyl ether used (trimethylolpropane triglycidyl ether, or BADGE) was reacted with both Cardolite® NC-700 and stearic acid, have a better T-Peel strength than the corresponding comparison composition ZRef2, which contains the same amount of a mixture of the reaction products between polyglycidyl ether and either only stearic acid or only Cardolite® NC-700, or a better T-Peel strength than the corresponding comparison composition ZRef3, in which contains the same amount of the mixture (corresponding to the adhesion promoter) of the reaction product between the polyglycidyl ether and only Cardolite® NC-700, i.e., without stearic acid, was reacted.

1. Compound of formula (I) 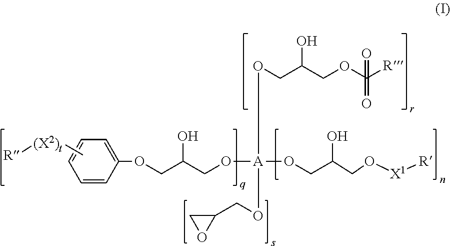
where
A is an (n+q+r+s)-hydric polyol A1 after removal of (n+q+r+s) hydroxyl groups,
X¹ is 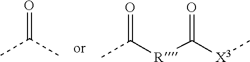
X² is 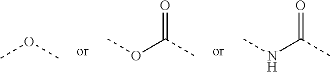
X³ is O or NH,
R′ is a branched, saturated, or unsaturated alkyl radical having 4 to 30 carbon atoms,
R″ is H or a branched, saturated, or unsaturated alkyl radical having 1 to 30 carbon atoms,
R′″ is an alkyl radical or an alkylphenyl radical having 6 to 30 carbon atoms,
R″″ is an aromatic, cycloaliphatic, or a branched, saturated, or unsaturated aliphatic divalent radical having 1 to 10 carbon atoms,
t has a value of 0 or 1,
n, q, r, and s each is an integer from 0 to 5, with the proviso that, of the values n, q, and r at least two are different from zero.

2. Compound according to claim 1, wherein the polyol A1 is an aliphatic, cycloaliphatic, or aromatic polyol.

3. Compound according to claim 1, wherein the polyol A1 is selected from the group consisting of pentaerythritol (=2,2-bis-(hydroxymethyl)-1,3-propanediol), dipentaerythritol (=3-(3-hydroxy-2,2-bis(hydroxymethyl)propoxy)-2,2-bis(hydroxymethyl)propan-1-ol), glycerol (=1,2,3-propane-triol), trimethylolpropane (=2-ethyl-2-(hydroxymethyl)-1,3-propanediol), trimethylolethane (=2-(hydroxymethyl)-2-methyl-1,3-propanediol), di(trimethylolpropane) (=2,2′-oxy-bis(ethylene)bis(2-ethylpropane-1,3-diol)), di(trimethylolethane) (=2,2′-oxy-bis(methylene)bis(2-methylpropane-1,3-diol)), diglycerol (=bis(2,3-dihydroxypropyl)ether), and triglycerol (=1,3-bis-(2,3-dihydroxypropyl)-2-propanol).

4. Thermosetting epoxy resin composition comprising
at least one epoxy resin EH with an average of more than one epoxy group per molecule;
at least one hardener B for epoxy resins, which is activated by elevated temperature; and
at least one compound of the formula (I) according to claim 1.

5. Thermosetting epoxy resin composition according to claim 4, wherein the proportion by weight of the compound of formula (I) is 0.001 to 20% by weight based on the weight of the thermosetting epoxy resin composition.

6. Thermosetting epoxy resin composition according to claim 4, wherein the proportion by weight of epoxy resin EH is 5 to 80% by weight based on the weight of the thermosetting epoxy resin composition.

7. Thermosetting epoxy resin composition according to claim 4, further comprising a toughness improver D selected from the group consisting of capped polyurethane polymers, liquid rubbers, epoxy resin-modified liquid rubbers, block copolymers, and core-shell polymers.

8. Thermosetting epoxy resin composition according to claim 4, further comprising a toughness improver D composed of liquid rubber which is an acrylonitrile/butadiene copolymer which is terminated with carboxyl groups or (meth)acrylate groups or epoxy groups, or is a derivative thereof.

9. Thermosetting epoxy resin composition according to claim 4, further comprising a toughness improver D which is a capped polyurethane polymer of the formula (IV) 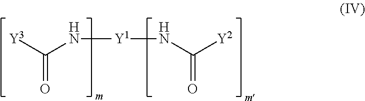
where
Y¹ is a linear or branched polyurethane polymer PU1 terminated with m+m′ isocyanate groups after the removal of all terminal isocyanate groups;
Y² is independently a capping group which is eliminated at a temperature of more than 100° C.;
Y³ is independently a group of the formula (IV′) 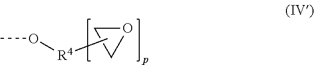
where R⁴ is a radical of an aliphatic, cycloaliphatic, aromatic or araliphatic epoxide containing a primary or secondary hydroxyl group after the removal of the hydroxyl and epoxy groups;
p=1, 2, or 3; and
m and m′ each has a value from 0 to 8, with the proviso that m+m′ is a value of 1 to 8.

10. Thermosetting epoxy resin composition according to claim 9, wherein Y² is a radical selected from the group consisting of 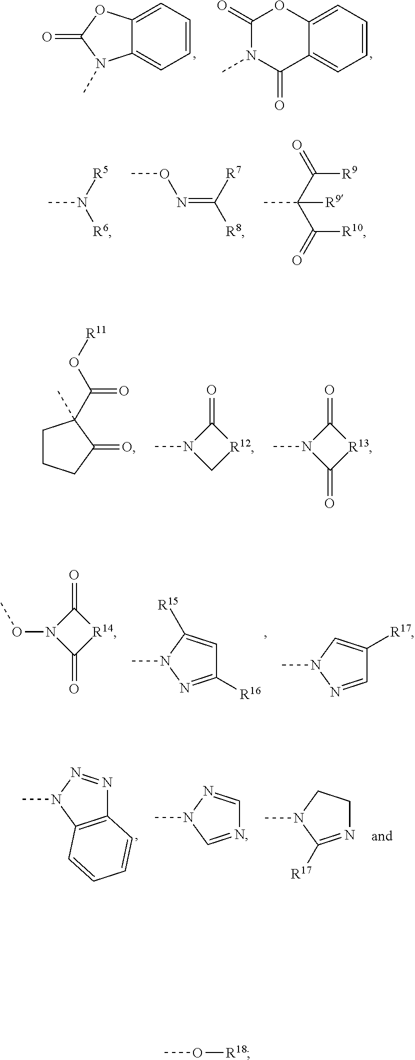
where
R⁵, R⁶, R⁷, and R⁸ are each independently an alkyl or cycloalkyl or aryl or aralkyl or arylalkyl group or R⁵ together with R⁶, or R⁷ together with R⁸, form part of a 4- to 7-membered ring which is optionally substituted;
R⁹, R^{9′}, and R¹⁰ are each independently an alkyl or aralkyl or aryl or arylalkyl group, or an alkyloxy or aryloxy or aralkyloxy group;
R¹¹ is an alkyl group;
R¹², R¹³, and R¹⁴ are each independently an alkylene group which has 2 to 5 carbon atoms and optionally has double bonds or is substituted, or a phenylene group or a hydrogenated phenylene group;
R¹⁵, R¹⁶, and R¹⁷ are each independently H or an alkyl group or an aryl group or an aralkyl group; and
R¹⁸ is an aralkyl group or a mono- or polycyclic, substituted, or unsubstituted aromatic group which optionally has aromatic hydroxyl groups.

11. Thermosetting epoxy resin composition according to claim 4, further comprising a reactive diluent G bearing epoxy groups.

12. Two-component epoxy resin composition comprising a resin component K1 and a hardener component K2,
the resin component K1 comprising at least one epoxy resin EH with an average of more than one epoxy group per molecule, and at least one compound of the formula (I) according to claim 1; and
the hardener component K2 comprising at least one polyamine B2 and/or polymercaptane B3.

13. A method of forming a cured composition comprising:
heating and subsequent crosslinking of the thermosetting epoxy resin composition according to claim 4.

14. An adhesive comprising the thermosetting epoxy resin composition according to claim 4.

15. Thermosetting epoxy resin composition according to claim 4, further comprising a chemical and/or physical blowing agent.

16. An adhesion promoter for a substrate S1, comprising a compound of formula (I).
